(19) United States
(12) Patent Application Publication
(10) Pub. No.: US 2006/0100180 A1
(43) Pub. Date: May 11, 2006

(54) Composition containing an androgenous 11b-halogen steroid and a progestational hormone, and male contraceptive based on said composition

(76) Inventors: Reinhard Nubbemeyer, Berlin (DE); Ursula Freiderike Habenicht, Berlin (DE); Rolf Bohlmann, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/522,169

(22) PCT Filed: Jul. 25, 2003
(86) PCT No.: PCT/EP03/08192

§ 371 Date: Nov. 16, 2005

Related U.S. Application Data
(60) Provisional application No. 60/449,400, filed on Feb. 25, 2003.

(30) Foreign Application Priority Data
Jul. 25, 2002 (DE) 102 34 525.2

(51) Int. Cl. A61K 31/56

(52) U.S. Cl. 514/170

(57) ABSTRACT

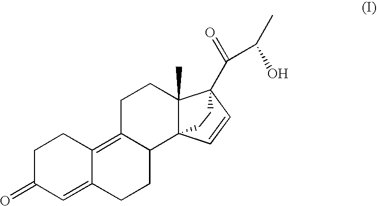

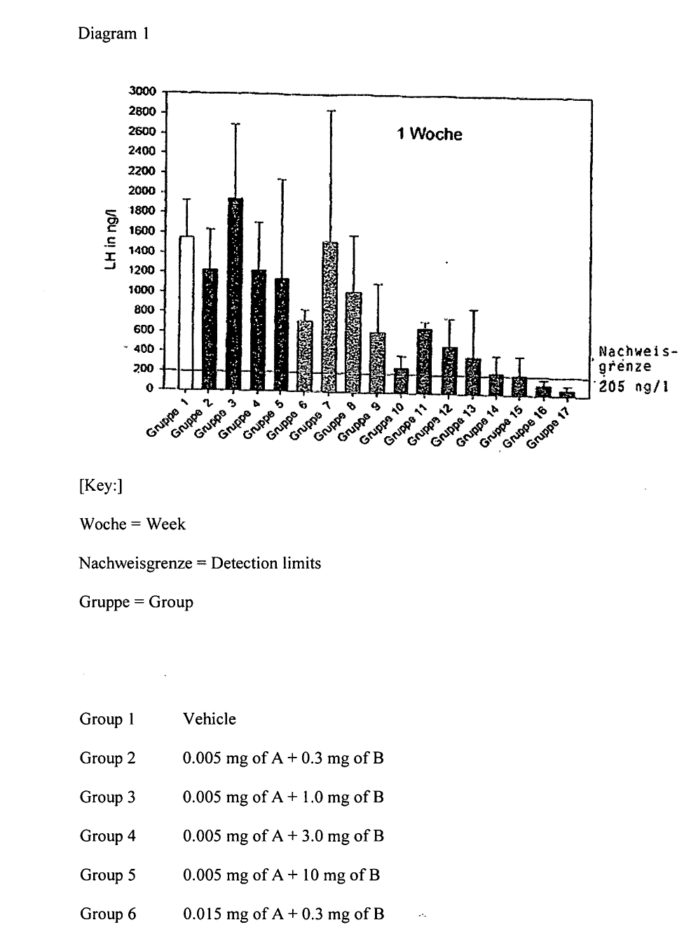

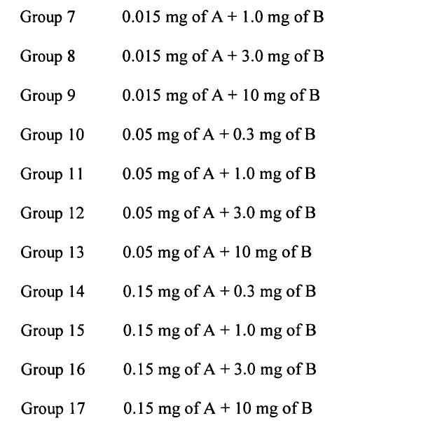

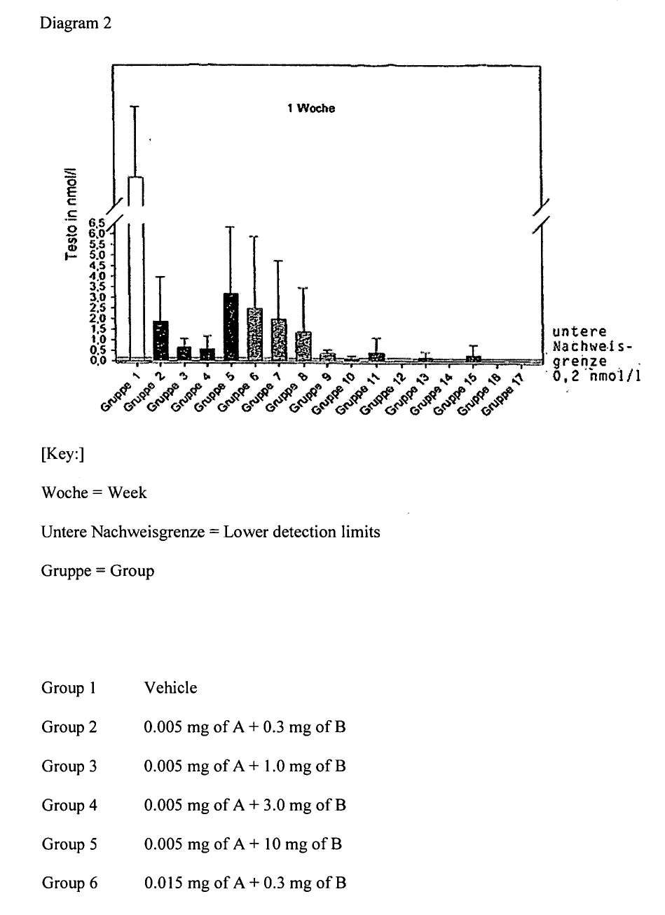

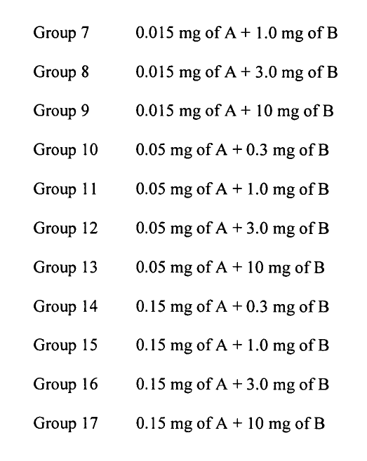

The invention relates to a novel chemical composition containing an androgenous 11β-halogen steroid and the progestational hormone of formula (I), to a pharmaceutical composition containing said chemical composition and to a male contraceptive based on said pharmaceutical composition.

This invention relates in the broader sense to a composition containing an androgenic 11β-halogen steroid, selected from the group of compounds of general formula I 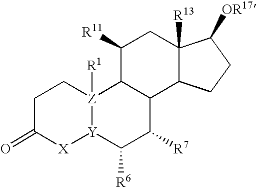
in which
X—Y-Z represents a group with one of the two structures CH═C—C or CH₂—C═C,
R¹ can be in α-position and β-position and stands for hydrogen, R or P-Q-R that is bonded via P to the basic ring structure, whereby P and Q represent straight-chain or branched-chain C₁- to C₈-alkylene, -alkenylene, or -alkinylene groups or their fluorinated derivatives and can be the same or different, and whereby R represents a CH₃ or CF₃ radical, provided that no substituent R¹ is present on Z if X—Y-Z represents the group CH₂—C═C,
R⁶ is a hydrogen atom or can have the meanings that are indicated under R⁷,
R⁷ stands for R or P-Q-R that is bonded via P to the basic ring structure, whereby these groups have the previously mentioned meanings,
R¹¹ represents a halogen,
R¹³ is methyl or ethyl, and
R^{17′} is hydrogen or stands for C(O)—R¹⁸, whereby
   R¹⁸ is a straight-chain or branched-chain C₁- to C₁₈-alkyl, -alkenyl, or -alkinyl radical or an aryl radical, or stands for T-U-V that is bonded via P to the C(O) group, whereby T and U represent straight-chain or branched-chain C₁- to C₁₈-alkylene, -alkenylene, -alkinylene groups, alicyclic C₃- to C₁₂ groups or aryl groups and are the same or different, and V is a straight-chain or branched-chain C₁- to C₁₈-alkyl-, -alkenyl- or -alkinyl radical or an aryl radical, or
   R¹⁸ has one of the previously mentioned meanings and in addition is substituted with one or more groups NR¹⁹R²⁰ or one or more groups SOₓR²¹, whereby x=0, 1 or 2, and R¹⁹, R²⁰ and R²¹ in each case are hydrogen or T-U-V that is bonded via T to N, S with the previously mentioned meaning, provided that in addition, the physiologically compatible addition salts with inorganic and organic acids are included,
   and the gestagen of the formula below.

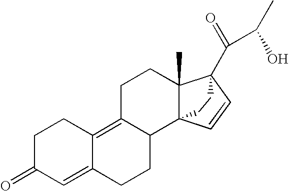

This composition is suitable for the production of pharmaceutical compositions. This invention therefore also relates to pharmaceutical compositions that contain the above-mentioned composition that consists of an androgenic 11β-halogen steroid and the gestagen of the formula 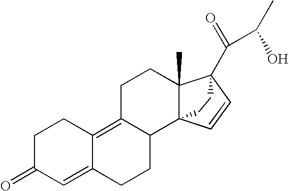
as well as a pharmacologically compatible vehicle and/or adjuvants.

Both in the composition and in the pharmaceutical composition, 11β-fluoro-17β-hydroxy-7α-methyl-estr-4-en-3-one is preferred as an androgenic 11β-halogen steroid.

In another embodiment, this invention relates to a male contraceptive agent based on the pharmaceutical composition above. According to another embodiment of the invention, 11β-fluoro-17β-hydroxy-7α-methyl-estr-4-en-3-one is contained in the male contraceptive agent as an androgenic 11β-halogen steroid.

In a special embodiment of this invention, both the androgenic 11β-halogen steroid and the gestagen are formulated in the male contraceptive agent such that both can be used in the form of a common implant or two separate implants in the body of the male user, so that the active compounds are released over an extended period to the organism of the user.

Continuous release of the gestagen over an extended period can also be achieved with a transdermal system, in which the gestagen is embedded.

It is also conceivable according to the invention to administer one of the active ingredients in an oral formulation and the other active ingredient as an implant or transdermally. It is also possible to administer both active ingredients orally. Finally, the possibility of administering one or both of the components of the composition according to the invention buccally or transmucosally can also be mentioned.

The concept for birth control in men is consistent with the global targets defined by WHO in “Reproduktive Gesundheit” [“Reproductive Health,” see WHO Task Force on Methods for the Regulation of Male Fertility (1990) Contraceptive Efficacy of Testosterone-Induced Azoospermia in Normal Men; Lancet 336: 955-959; WHO Task Force on Methods for the Regulation of Male Fertility (1993) Comparison of Two Androgens Plus Depot-Medroxyprogesterone Acetate for Suppression to Azoospermia in Indonesian Men; Fertil Steril [Fertile Sterile] 60: 1062; WHO Task Force on Methods for the Regulation of Male Fertility (1995) Rate of Testosterone-Induced Suppression to Severe Oligozoospermia or Azoospermia in Two Multinational Clinical Studies; Int J Androl 18: 157-165; WHO Task Force on Methods for the Regulation of Male Fertility (1996) Contraceptive Efficacy of Testosterone-Induced Azoospermia and Oligozoospermia in Normal Men; Fertil Steril 65; 821-829]. Integral components of this strategy are contraceptive agents for men and women. Since in particular contraceptive methods for males are still lacking, the development of such is regarded as absolutely necessary (Andrologie, Grundlagen und Klinik der reproductiven Gesundheit des Mannes [Andrology, Principles and Male Reproductive Health Clinic]; Editors E. Nieschlag, H. M. Behre; Springer-Verlag, 2^{nd} Edition, page 442 ff, 2000). Farthest advanced in development are hormonal approaches to male birth control. They are distinguished by proven reversibility and effectiveness.

Hormonal male contraception is based on the suppression (the stopping) of spermatogenesis, which ultimately results in azoospermia and thus in male infertility. Mechanistically, the two gonadotropins LH (luteinizing hormone) and FSH (follicle-stimulating hormone) are significantly inhibited, i.e., the serum concentrations of these two hormones are no longer detectable. As a result of the LH suppression, the testicular testosterone production is also inhibited (both hormones belong to an endocrine control circuit). The deficit of all three hormones is necessary to inhibit the spermatogenesis. The essential drawback of the described method is the androgen deficiency and the symptoms/consequences resulting therefrom for males.

Methods for male contraception attempt to suppress LH, FSH and intratesticular testosterone and thus to prevent spermatogenesis, while peripheral testosterone is substituted by an androgen that is fed exogenically. As an androgen, testosterone or testosterone ester (e.g., testosterone enanthate, testosterone buciclate) was previously used. The object of endocrine testosterone exists in upholding the libido, the potency, male behavior, protein metabolism, erythropoiesis and other functions, such as mineral and bone metabolism.

In short, the purpose consists in dropping the testosterone in the testes to a level as is found in peripheral blood, while the levels in the general circulation are to be upheld.

The suppression of spermatogenesis by testosterone or testosterone esters alone, which seemed to be an ideal contraceptive agent at first, has not yet proven efficient enough, however, and in some cases it took too long for a reliable action to set in (onset of up to 6 months). Morever, ethnic differences were also noted. Doses that are too high showed significant and undesirable side effects.

With a treatment with testosterone, it was shown that side effects develop, in particular an enlargement of the prostate owing to an increase in the number of cells and glands of the stroma (BPH: benign prostate hyperplasia). With the 5α-reductase-mediated metabolism of testosterone, dihydrotestosterone (DHT), which, i.a., can lead to the occurrence of BPH, is produced (Cummings et al., ibid.; WO 99/13883 A1).

Testosterone is not available in oral form at this time, therefore alternative dispensing forms (i.m, patches, etc.) must be used.

To accelerate the onset and also to improve efficiency, testosterone was combined with other gonadotropin-suppressing substances, with GNRH antagonists (GnRH=gonadotropin releasing hormone). The rate of azoospermia as well as the time of the onset were clearly improved with this combination. The currently available GnRH antagonists, however, must be administered daily (i.m. or s.c., oral administration is not available), and their production is expensive. This combination is therefore not attractive.

The use of either the progestin cyproterone acetate or levonorgestrel was either ineffective in the suppression of spermatogenesis or in higher dosages resulted in a significant drop in the number of red blood cells (Merrigiola et al., 1998; Merrigiola et al., 1997; Merrigiola et al., 1996; Bebb et al., 1996).

The use of a mixture of two compounds, an estrogen with an estrogen, in combination is described in U.S. Pat. No. 4,210,644.

A method that aims at the inhibition of spermatogenesis by percutaneous or oral administration of testosterone and the oral administration of norethisterone acetate was also described (Guerin and Rollet; 1998). To achieve an azoospermia, however, fairly high doses of the two components are necessary.

For the replacement of testosterone for male contraception, 7α-methyl-19-nortestosterone (MeNT) was proposed, which has, on the one hand, a higher biological effectiveness than testosterone, since it has a higher binding affinity to the androgen receptors. On the other hand, because of a steric inhibition by the 7α-methyl group, it presumably resists metabolization by 5α-reductase (Cummings et al., ibid., WO 99/13883 A1, WO 99/13812 A1, U.S. Pat. No. 5,342,834).

Owing to the last-mentioned property, a clearly more advantageous side-effect profile in comparison to testosterone is expected, especially with respect to the prostate.

A combination of 7α-methyl-19-nortestosterone with a gestagen cannot be found in these sources.

Other compounds that are comparable to 7α-methyl-19-nortestosterone in their selective androgen action are the androgenic 11β-halogen steroids of general formula I, in particular the 11β-fluoro-17β-hydroxy-7α-methyl-estr-4-en-3-one, that are to be used according to the invention.

These compounds are described for the first time in DE 101 04 327.9. The compounds have an improved metabolic stability compared to 7α-methyl-19-nortestosterone. DE 101 04 327.9 is a non-prepublished document.

For description and definition of the substituents of the compounds of general formula I, reference is made to this document.

The compounds are proposed for use in male contraception. They can be used together with gestagens without stating more specifically what gestagens are meant in this case.

The object of this invention is to make available a male contraceptive agent based on androgen/gestagen that does not use testosterone as androgen. At the same time, the dose of the androgen to be used is to be decreased by the gestagen, and thus side effects are reduced.

This object is achieved by the above-mentioned combination of an androgenic 11β-halogen steroid, in particular 11β-fluoro-17β-hydroxy-7α-methyl-estr-4-en-3-one, with the gestagen of formula

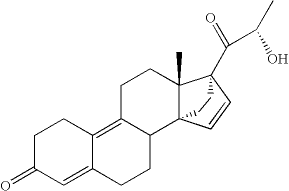

This gestagen is described in International Patent Application WO 96/20209 (DE 44 47 401.6). Joint administration with an androgen to achieve male infertility cannot be found in this application. This is a greatly effective gestagen after oral administration. Other routes of administration were also proposed in this application, however. In addition, a transdermal system that contains this gestagen is described in Patent Application EP 00250449.6.

With the application of the above-mentioned combination as a male contraceptive agent, an adequate inhibition of the sperm production in the testes can be achieved with a relatively low substitution dose of the androgen at the same time. In this connection, a synergistic effect is achieved.

By means of the composition according to the invention as a male contraceptive agent, it is possible, with low dosages of both components, to push the LH, FSH and testosterone parameters into the range where they are not detected or are no longer effective. The drops in the LH and FSH parameters occur together.

For reliability and acceptance of the contraceptive agent according to the invention by men, in this case it is of decisive importance that the drop of parameters, decisive for the safety of the contraceptive agent, be accomplished relatively quickly. The “onset” for the contraceptive agent according to the invention is about 3 months after the beginning of use.

The period of use of the contraceptive agent according to the invention can in principle and optionally be unlimited, i.e., no more contraception is required by the user.

In contrast, the contraceptive agent according to the invention always allows the user to recover fertility.

The dosages of the androgenic 11β-halogen steroid of general formula I, in particular 11β-fluoro-17β-hydroxy-7α-methyl-estr-4-en-3-one and the gestagen, are selected such that at the latest 3 months after the beginning of use, the levels of LH, testosterone and FSH lie in the range where these parameters are no longer effective.

For 11β-fluoro-17β-hydroxy-7α-methyl-estr-4-en-3-one, a daily effective amount of 0.7 μg to 1.5 μg, preferably 0.7 μg to 1.0 μg, is adequate.

In determining an effective amount of the androgenic steroid of Formula I, it can be considered that 11β-fluoro-17β-hydroxy-7α-methyl-estr-4-en-3-one is about 10× more effective than testosterone.

In the case of application by means of an implant or another system that releases the active ingredient over an extended period, the latter must be constituted so that the indicated amount is released daily.

As a guiding rule for the dosage of the gestagen that is to be used according to the invention, it may hold true that in terms of the inhibition of spermatogenesis, the selected amount has an effect comparable to that of a daily dose of 200 μg to 300 μg of levonorgestrel. An equieffective amount of a daily oral administration of 240 μg to 260 μg of levonorgestrel is preferred.

The determination of equieffective amounts of levonorgestrel and the gestagen that is to be used according to the invention is carried out according to methods that are known to one skilled in the art, for example in the pregnancy maintenance test in rats.

For formulation of the two active ingredients in the contraceptive agent according to the invention, reference is made to the above-mentioned citations, in which the active ingredients themselves are described. Techniques for the formulation of androgens or gestagens for long-lasting release of these active ingredients are known in the prior art, thus, e.g., the implant system Norplant or Jardelle for gestagens.

In addition, for formulation of the gestagen to be used according to the invention, reference is made to WO 00/21570 (formulation with a cyclodextrin) and WO 02/49622 (transdermal system that contains the gestagen that is to be used according to the invention).

The determination of the parameters LH, FSH as well as testosterone is carried out according to known methods.

The effectiveness of the combination according to the invention is confirmed by
The determination of the LH concentration in the serum in juvenile male rats after a treatment period of 1 week with s.c. administration of a combination of compounds A and B (Diagram 1) as well as
The determination of the testosterone concentration in the serum in adult male rats after a treatment period of 1 week with s.c. administration of a combination of compounds A and B (Diagram 2).

In both cases, as early as after one week, these parameters lie below the detection limit (rapid onset).

Compound A is 11β-fluoro-17β-hydroxy-7α-methyl-estr-4-en-3-one, and Compound B is the gestagen to be used according to the invention.

The indicated doses were administered per kg of body weight daily.

In further tests on male rats, it was possible to show that with the combination according to the invention (compound A with compound B; identical dosages as indicated in Diagrams 1 and 2) after/at a six-week treatment period:
The relevant organ weights (prostate, epididymis, seminal vesicles and testes) were decreased depending on hormone status;
The sperm count is reduced to less than 10% of the control value;
The values of hormone LH and testosterone always remain under the detection limits even after a treatment that takes place over this period (i.e., both a rapid onset, see above, and enduring action).

### BIBLIOGRAPHIC REFERENCES

E. Nieschlag and H_M_Behre; Testosterone in Male Contraception. In E. Nieschlag and H. M. Behre, eds. Testosterone: Action, Deficiency, Substitution, 1998, Springer, Berlin, p p. 513-528.
M. C_Merrigiola, W. J. Bremner, A. Constantino, A. Pavani, M. Capelli and C. Flamigni; Low Dose of Cyproterone Acetate and Testosterone Enanthate for Contraception in Men_, Hum Reprod., (1998) 13, 1225-1229.
M. C. Merrigiola, W. J. Bremner, A. Constantino, A. Pavant, M. Capelli and C. Flamigni, An Oral Regimen of Cyproterone Acetate and Testosterone Undecanoate for Spermatogenic Suppression in Men, Ferld. Steril. (1997). 68, 84-850.
M. C. Merrigiola, W. J_Bremner, C. A. Paulsen, A. Valdiserri, L. Incorvaia, R. Motta, A. Pavani, M. Capelli and C. Flamigni, A Combined Regimen of Cyproterone Acetate and Testosterone Enanthate as a Potentially Highly Effective Male Contraceptive, J. Clin. Endocrinol. (1996). 81, 3018-3023
R. A. Bebb, B. D. Anawalt, R. B. Christensen, C. A. Paulsen, W. J. Bremner and A. M. Matsumoto., Combined Administration of Levonorgestrel and Testosterone Induces More Rapid and Effective Suppression of Spermatogenesis than Testosterone Alone, A Promising Contraceptive Approach, J. Clin Endocriniol. Metab_(1996) 81, 757-762.
J. F. Guenn and J. Rollet, International Journal of Andrology, 1988, 11, pp. 187-199.
Hadgraft and Guy; Transdermal Drug Delivery; Developmental Issues and Research Initiatives, Marcel Dekker, Inc., 1989.
M. Ottel and E. Schillinger (editors), Handbook of Experimental Pharmacology, Vol. 135/11, Androgens and Antiestrogens/1, Pharmacology and Clinical Applications of Estrogens and Antiestrogens; K_-H. Fritzemeier and C. Hegele-Hartung. In Vitro and In Vivo Models to Characterize Estrogens and Antiestrogens; Springer-Verlag, Berlin. Heidelberg, 1999.
F. Neuman, F. Daher, J. Brotherton, K.-J. Graf, S. H. Hasan, H. J. Horn, A. Hughes, G. W. Oertel, H. Steinheck, H. E. Voss, R. K. Wagner, Androgens II and AntiAndrogens, Springer-Verlag, Berlin, Heidelberg, 1974.
Fuhrmann, Bengston, Repenthin, and Schillinger, J. Steroid Biochem. Mol. Biol., 1992, 42(8), 787).
Lancet 336; 1990, 955-959
Fertil Steril 60; 1993, 1062
Int J Androl 18; 1995; 157-165
Fertil Steril 65; 1996, 821-829
Andrologie, Grundlagen und Klinik der reproductiven Gesundheit des Mannes; Editors E. Nieschlag, H. M. Behre; Springer-Verlag, 2^{nd} Edition, page 442 ff, 2000.

1. Composition containing an androgenic 11β-halogen steroid, selected from the group of compounds of general formula I 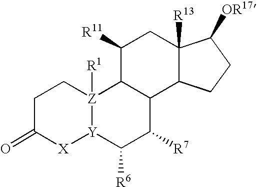
in which
X—Y-Z represents a group with one of the two structures CH═C—C or CH₂—C═C,
R¹ can be in α-position and β-position and stands for hydrogen, R or P-Q-R that is bonded via P to the basic ring structure, whereby P and Q represent straight-chain or branched-chain C₁- to C₈-alkylene, -alkenylene, or -alkinylene groups or their fluorinated derivatives and can be the same or different, and whereby R represents a CH₃ or CF₃ radical, provided that no substituent R¹ is present on Z if X—Y-Z represents the group CH₂—C═C,
R⁶ is a hydrogen atom or can have the meanings that are indicated under R⁷,
R⁷ stands for R or P-Q-R that is bonded via P to the basic ring structure, whereby these groups have the previously mentioned meanings,
R¹¹ represents a halogen,
R¹³ is methyl or ethyl, and
R^{17′} is hydrogen or stands for C(O)—R¹⁸, whereby
R¹⁸ is a straight-chain or branched-chain C₁- to C₁₈-alkyl, -alkenyl, or -alkinyl radical or an aryl radical, or stands for T-U-V that is bonded via P to the C(O) group, whereby T and U represent straight-chain or branched-chain C₁- to C₁₈-alkylene, -alkenylene, -alkinylene groups, alicyclic C₃- to C₁₂ groups or aryl groups and are the same or different, and V is a straight-chain or branched-chain C₁- to C₁₈-alkyl-, -alkenyl- or -alkinyl radical or an aryl radical, or
R¹⁸ has one of the previously mentioned meanings and in addition is substituted with one or more groups NR¹⁹R²⁰ or one or more groups SOₓR²¹, whereby x=0, 1 or 2, and R¹⁹, R²⁰ and R²¹ in each case are hydrogen or T-U-V that is bonded via T to N, S with the previously mentioned meaning, provided that in addition, the physiologically compatible addition salts with inorganic and organic acids are included,
and the gestagen of the formula below.

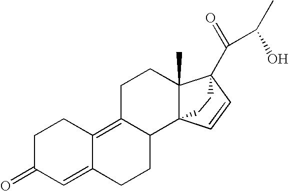

2. Composition according to claim 1, characterized in that the 11β-halogen steroid of general formula I is the compound 11β-fluoro-17β-hydroxy-7α-methyl-estr-4-en-3-one.

3. Pharmaceutical composition containing a composition according to claim 1 as well as a pharmaceutically compatible vehicle and/or adjuvants.

4. Pharmaceutical composition according to claim 3, wherein the 11β-halogen steroid of general formula I is the compound 11β-fluoro-17β-hydroxy-7α-methyl-estr-4-en-3-one.

5. Male contraceptive agent containing a pharmaceutical composition according to claim 3.

6. Male contraceptive agent containing a pharmaceutical composition according to claim 4.

7. Male contraceptive agent according to claim 5, wherein the androgenic compound of general formula I is formulated pharmaceutically such that the latter can be implanted in the body of the male user over an extended period such that the androgenic compound is released continuously over this extended period to the organism of the user.

8. Male contraceptive agent according to claim 5, wherein the androgenic compound of general formula I is provided therein for oral administration.

9. Male contraceptive agent according to claim 5, wherein the gestagen therein is formulated pharmaceutically such that it is released to the body of the male user over an extended period.

10. Male contraceptive agent according to claim 9, wherein the gestagen is formulated such that the latter can be implanted in the body of the male user over an extended period, such that the gestagen is released continuously over this extended period to the organism of the user.

11. Male contraceptive agent according to claim 10, wherein the gestagen is formulated in a transdermal system.

12. Male contraceptive agent according to claim 5, wherein the gestagen is formulated for oral administration.
